(19) United States
(12) Patent Application Publication
(10) Pub. No.: US 2025/0110132 A1
(43) Pub. Date: Apr. 3, 2025

(54) BIOMARKERS FOR THE DETERMINATION OF SAMPLE ADEQUACY AND LUNG CANCER METASTASES

(71) Applicant: UNIVERSITY HEALTH NETWORK, Toronto (CA)

(72) Inventors: Kazuhiro YASUFUKU, Toronto (CA); Tsukasa ISHIWATA, Toronto (CA); Andrew SAGE, Toronto (CA); Shafique KESHAVJEE, Toronto (CA); Kosuke FUJINO, Kumamoto City (JP); Takahiro NAKAJIMA, Chiba (JP)

(21) Appl. No.: 18/854,884

(22) PCT Filed: Apr. 6, 2023
(86) PCT No.: PCT/CA2023/050473

§ 371 Date: Oct. 7, 2024

Related U.S. Application Data
(60) Provisional application No. 63/328,527, filed on Apr. 7, 2022.

(51) Int. Cl. G01N 33/574; C12Q 1/6886; G01N 33/543; G01N 33/58

(52) U.S. Cl. CPC G01N 33/57423; C12Q 1/6886; G01N 33/54388; G01N 33/582; C12Q 2600/158; G01N 2333/521

(57) ABSTRACT

The present disclosure provides methods to assess lymph node samples such as endobronchial ultrasound-guided transbronchial needle aspiration (EBUS-TBNA) samples to determine sample sufficiency and/or to detect metastasis in mediastinal lymph nodes using lymph node biomarkers. Also provided are devices and kits that can be used to perform the methods disclosed herein.

### RELATED APPLICATIONS

This PCT application claims the benefit of priority of U.S. Provisional Patent Application 63/328,527 filed Apr. 7, 2022, which is incorporated by reference in its entirely.

### FIELD

The present disclosure relates to assessment of biopsy samples such as endobronchial ultrasound-guided transbronchial needle aspiration (EBUS-TBNA) samples using biomarkers to determine sample sufficiency and/or to detect metastasis in mediastinal lymph nodes.

### BACKGROUND

Lung cancer is the most common cancer and the leading cause of cancer death; lung cancer is responsible for the deaths of 1.6 million worldwide.^{1,2} Although it is one of the most aggressive malignant tumors, as with many other types of cancer, patients can live cancer-free if the diagnosis is accurate and timely treatment is administered.

Endobronchial ultrasound-guided transbronchial needle aspiration (EBUS-TBNA) is a highly sensitive, minimally-invasive procedure³ and is universally accepted as one of the recommended first steps in mediastinal staging of lung cancer.⁴ EBUS-TBNA uses a thin, flexible bronchoscope coupled with an ultrasound probe to enable the clinician to locate and biopsy lymph nodes in real time, and thus provide a more accurate assessment of the patient's condition.^{5,6} EBUS-TBNA technology has become an essential tool for respirologists and thoracic surgeons in over 2,500 cancer centres worldwide. To achieve optimal EBUS-TBNA mediastinal lymph node assessment, rapid on-site cytologic evaluation (ROSE) is beneficial in providing immediate biopsy evaluation.^{7,8} ROSE provides fast and accurate feedback to the clinical team-specifically whether a sufficient amount of biopsy is obtained and if signs of metastasis are observed, in which case the bronchoscopist can cease further EBUS-TBNA lymph node sampling (FIG. 1). Unfortunately, the availability of ROSE is not widespread, which creates disparities both within and across institutions. As a result, many EBUS-TBNA procedures are performed without ROSE which leads to an increased frequency of non-diagnostic specimens for pathological evaluation and the subsequent need to repeat bronchoscopy.

The development of rapid point-of-care platforms for EBUS-TBNA can aid in sample assessment. These technologies enable the accurate detection of adequate biopsy specimens and allow for the accurate detection of metastasis in mediastinal lymph nodes.

### SUMMARY

As demonstrated herein, the levels of the biomarkers CXCL13 and/or CCL21 can be used to determine lymph node, optionally EBUS-TBNA, sample sufficiency. In addition, the disclosure also demonstrates that one or more biomarkers selected from the group consisting of EpCAM, KRT19, SFN, KRT7, INSM1 and KRT5 can be used in the detection of metastasis in mediastinal lymph nodes. Accordingly, provided in various aspects are methods and devices which can be used to assess lymph node, optionally EBUS-TBNA, samples to determine sample sufficiency and/or detection of metastasis.

An aspect of the present disclosure is a method of determining lymph node sample sufficiency, the method comprising: (a) providing a lymph node sample obtained from a subject; and (b) measuring a level of one or more lymph node sufficiency biomarkers selected from CXCL13 and/or CCL21 in the sample; wherein the level of the one or more lymph node sufficiency biomarkers is indicative of sample sufficiency.

In an embodiment, the method further comprises comparing the level of the one or more lymph node sufficiency biomarkers to a pre-determined cut-off value or set of predetermined cut-off values.

In an embodiment the predetermined cut-off value is determined from a plurality of sufficient samples.

In an embodiment, the method further comprises providing a subsequent lymph node sample and repeating step (b) when the level of the one or more sufficiency biomarkers in a previous sample, such as an immediately preceding sample, indicates sample insufficiency.

In an embodiment, the lymph node sample and the subsequent samples are taken from a same lymph node.

In an embodiment, the one or more lymph node sufficiency biomarkers is or comprises CXCL13.

In an embodiment, the one or more lymph node sufficiency biomarkers is or comprises CCL21.

In an embodiment, one or more lymph node sufficiency biomarkers further comprises SIGLEC1.

In an embodiment, the one or more lymph node sufficiency biomarkers further comprises UBD.

In an embodiment, the one or more lymph node sufficiency biomarkers is or comprises CXCL13 and CCL21.

In an embodiment, the one or more lymph node sufficiency biomarkers is or comprises CXCL13, CCL21 and SIGLEC1.

In an embodiment, the one or more lymph node sufficiency biomarkers is or comprises CXCL13, CCL21 and UBD.

In an embodiment, the one or more lymph node sufficiency biomarkers is or comprises CXCL13, CCL21, SIGLEC1 and UBD.

In an embodiment, the sample is a biopsy sample.

In an embodiment, the biopsy sample is a needle aspirate sample.

In an embodiment, the needle aspirate sample is an endobronchial ultrasound-guided transbronchial needle aspiration (EBUS-TBNA) sample.

In an embodiment, for a sample determined to be sufficient, the method further comprises subjecting the sample to an assay, optionally next generation sequencing, ROSE or a pathological assay and/or assessment.

In an embodiment, the method further comprises performing an assay to detect metastasis.

In an embodiment, the assay for detecting metastasis comprises measuring a level of one or more lymph node metastasis biomarkers selected from EpCAM, KRT19, SFN, and/or KRT7 in the sample; wherein the level of the one or more lymph node metastasis biomarkers is indicative of metastasis.

In an embodiment, the group of lymph node metastasis biomarkers further comprises INSM1 and KRT5.

In an embodiment, the one or more lymph node metastasis biomarkers are at least 2 metastasis biomarkers.

In an embodiment, the one or more lymph node metastasis biomarkers are at least 3 lymph node biomarkers for metastasis.

In an embodiment, the one or more lymph node metastasis biomarkers are at least 4 lymph node biomarkers for metastasis.

In an embodiment, the one or more lymph node metastasis biomarkers are at least 5 lymph node biomarkers for metastasis.

In an embodiment, the one or more lymph node metastasis biomarkers are the 6 lymph node biomarkers for metastasis.

In an embodiment, the one or more lymph node metastasis biomarkers is or comprises EpCAM.

In an embodiment, the one or more lymph node metastasis biomarkers is or comprises KRT19.

In an embodiment, the one or more lymph node metastasis biomarkers is or comprises SFN.

In an embodiment, the one or more lymph node metastasis biomarkers is or comprises KRT7.

In an embodiment, the one or more lymph node metastasis biomarkers is or comprises INSM1.

In an embodiment, the one or more lymph node metastasis biomarkers is or comprises KRT5.

In an embodiment, the method further comprises comparing the level of the one or more lymph node metastasis biomarkers to a predetermined cut-off value or set of predetermined cut-off values.

In an embodiment, the predetermined cut-off value or set of predetermined cut-off values is determined from a plurality of non-metastatic lymph node samples.

In an embodiment, the method is followed by a method for detecting meta, when the sample is determined to be sufficient.

In an embodiment, the determining sample sufficiency and detecting metastasis are performed concurrently.

In an embodiment, the sample is from a subject suspected of having lung cancer.

In an embodiment, the sample is from a subject diagnosed as having lung cancer.

In an embodiment, the lung cancer is adenocarcinoma, non-small cell lung cancer, small cell lung cancer or squamous cell lung cancer.

In an embodiment, the sample is a protein fraction.

In an embodiment, the level of the one or more lymph node biomarkers is measured using in an affinity assay using a binding agent.

Another aspect provides a method of detecting metastasis, the method comprising: providing a lymph node sample obtained from a subject, measuring a level of one or more lymph node metastasis biomarkers selected from the group consisting of EpCAM, KRT19, SFN, and KRT7 in the sample; wherein the level of the one or more lymph node metastasis biomarkers is indicative of metastasis.

In an embodiment, the group of lymph node metastasis biomarkers further comprises INSM1 and KRT5.

In an embodiment, the one or more lymph node metastasis biomarkers are at least 2 lymph node biomarkers for metastasis.

In an embodiment, the one or more lymph node metastasis biomarkers are at least 3 lymph node biomarkers for metastasis.

In an embodiment, the one or more lymph node metastasis biomarkers are at least 4 lymph node biomarkers for metastasis.

In an embodiment, the one or more lymph node metastasis biomarkers are at least 5 lymph node biomarkers for metastasis.

In an embodiment, the one or more lymph node metastasis biomarkers are the 6 lymph node biomarkers for metastasis.

In an embodiment, one or more lymph node metastasis biomarkers is or comprises EpCAM.

In an embodiment, the one or more lymph node metastasis biomarkers is or comprises KRT19.

In an embodiment, the one or more lymph node metastasis biomarkers is or comprises SFN.

In an embodiment, the one or more lymph node metastasis biomarkers is or comprises KRT7.

In an embodiment, the one or more lymph node metastasis biomarkers is or comprises INSM1.

In an embodiment, the one or more lymph node metastasis biomarkers is or comprises KRT5.

In an embodiment, the method further comprises comparing the level of the one or more lymph node metastasis biomarkers to a predetermined cut-off value or set of predetermined cut-off values.

In an embodiment, the predetermined cut-off value or set of predetermined cut-off values is determined from a plurality of non-metastatic lymph node samples.

In an embodiment, sample is a biopsy sample.

In an embodiment, the biopsy sample is a needle aspirate sample.

In an embodiment, the needle aspirate sample is an endobronchial ultrasound-guided transbronchial needle aspiration (EBUS-TBNA) sample.

In an embodiment, the sample is from a subject suspected of having lung cancer.

In an embodiment, the sample is from a subject diagnosed as having lung cancer.

In an embodiment, the lung cancer is adenocarcinoma, non-small cell lung cancer, small cell lung cancer or squamous cell lung cancer.

In an embodiment, the sample is a protein fraction.

In an embodiment, the level of the one or more lymph node biomarkers is measured using in an affinity assay using a binding agent.

In an embodiment, the sample is a nucleic acid sample.

In an embodiment, the level of the one or more lymph node biomarkers is measured using a hybridization assay using a nucleic acid or peptide nucleic acid probe.

A further aspect is a device for measuring the level of one or more lymph node sufficiency biomarkers selected from the group consisting of CXCL13, CCL21, and optionally SIGLEC1 and UBD, for determining sample sufficiency.

In an embodiment, the device is a point of care device.

In an embodiment, the device is further for measuring the level of one or more lymph node metastasis biomarkers selected from the group consisting of EpCAM, KRT19, SFN, KRT7, INSM1 and KRT5 for detecting metastasis.

In an embodiment, the device comprises an immunological assay.

In an embodiment, the immunological assay is an enzyme-linked immunosorbent assay (ELISA) or a lateral flow assay (LFA).

In an embodiment, the immunological assay is a lateral flow assay (LFA).

In an embodiment, the device comprises:
a. a sample pad for loading a sample on the device;
b. a conjugate pad, comprising binding agents for each of the one or more lymph node biomarkers;
c. a reaction zone for capturing lymph node biomarkers conjugated to binding agents;
d. a control zone, comprising a positive control; and
e. a wicking zone for driving flow of the sample through the device;
wherein the sample after being loaded onto the sample pad flows through (i) the conjugate pad, (ii) the reaction zone, and (iii) the control zone into the wicking zone.

In an embodiment, the binding agents are fluorescently labelled.

In an embodiment, the device is for use in the method described herein.

A further aspect provides a device, optionally a point-of-care (POC) device, for measuring the level of one or more lymph node metastasis biomarkers selected from the group consisting of EpCAM, KRT19, SFN, KRT7, INSM1 and KRT5, for detecting metastasis.

In an embodiment, the device comprises a PCR-based assay.

In an embodiment, the device comprises an immunological assay.

In an embodiment, the immunological assay is an enzyme-linked immunosorbent assay (ELISA) or lateral flow assay (LFA).

In an embodiment, the immunological assay is a lateral flow assay (LFA).

In an embodiment, the device comprises:
a. a sample pad for loading a sample on the device;
b. a conjugate pad, comprising binding agents for each of the one or more lymph node metastasis biomarkers;
c. a reaction zone for capturing lymph node metastasis biomarkers conjugated to binding agents;
d. a control zone, comprising a positive control; and
e. a wicking zone for driving flow of the sample through the device;
   wherein the sample after being loaded onto the sample pad flows through (i) the conjugate pad, (ii) the reaction zone, and (iii) the control zone into the wicking zone.

In an embodiment, the binding agents are fluorescently labelled.

In an embodiment, the device comprises an electrochemical assay.

In an embodiment, the device is for use in the method for detecting metastasis described herein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the present disclosure will now be described in relation to the drawings in which:

FIG. 1 shows an example of an EBUS-TBNA clinical workflow.

FIGS. 2A-2F are a series of graphs that shows the lymph node biomarkers CXCL13 and CCL21 predict sufficient EBUS-TBNA samples. CXCL13 (A) and CCL21 (D) mRNA expression levels in lymph node samples compared to samples of whole blood. Protein levels for CXCL13 (B) and CCL21 (E) in sufficient and insufficient samples as determined by ROSE. Protein levels for CXCL13 (C) and CCL21 (F) according to the final cytopathology report results. Dashed line represents the CXCL13 (2 pg/mL) and CCL21 (170 pg/mL) cutoffs. p-values are indicated in each panel.

FIG. 3 is a graph that shows qRT-PCR analysis of EBUS-TBNA lymph node (LN) biopsy gene expression for 12 biomarkers measured in n=24 metastatic and n=10 non-metastatic EBUS-TBNA samples.

FIG. 4 is a graph that shows expression of EpCAM, KRT19, SFN, and KRT7 in 14 lung cancer cell lines (5 ADCs, 4 SQCs, and 5 SCLC) and 5 normal lungs (NL).

FIG. 5 is a series of images of immunohistochemical staining of EBUS-TBNA (KRT19, KRT7, EpCAM, SFN, KRT5, and INSM1) in surgically resected ADC, SQC, SCLC, and negative lymph node biopsies.

FIG. 6 is a graph that shows diagnostic classification of EBUS-TBNA lymph node biopsies for KRT19, KRT7, EpCAM, SFN, KRT5, and INSM1 from n=79 ADCs, n=55 SQCs, n=55 SCLCs, and n=133 normal lymph nodes (NL). Cases with more than 10% immunoreactivity were considered as positive cases.

FIG. 7 is an illustration of rapid detection of lymph node biomarkers using a point-of-care (POC) device. An EBUS-TBNA biopsy sample is obtained for biomarker detection (left panel). Microchips are functionalized with complementary nucleic acid sequences that specifically report on the presence or absence of target nucleic acids (middle panel). The negatively charged phosphate backbone of the target sequences attracts a positively charged electrochemical reporter molecule (E) which can generate a measurable current.

FIGS. 8A-8B are a series of graphs that show rapid detection of lymph node biomarkers using a POC device. (A) Lower limit of detection determination for EpCAM sensors challenged with complementary EpCAM DNA. (B) Comparison of EBUS-TBNA microchip sensor output to qRT-PCR in a single adenocarcinoma biopsy sample.

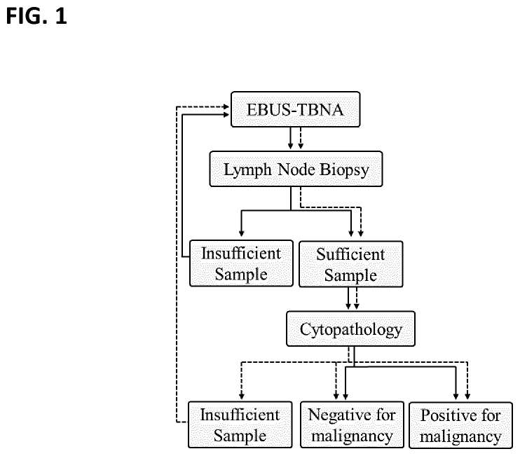

### DETAILED DESCRIPTION OF THE DISCLOSURE

The present disclosure is related to methods and devices for rapid assessment of lymph node and particularly, EBUS-TBNA samples to determine sufficiency and/or detect of metastasis, in for example mediastinal lymph nodes, by measuring the level of one or more biomarkers described herein.

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

The term “subject” also referred as patient, as used herein includes all members of the animal kingdom including mammals, and suitably refers to humans.

As used herein, the terms “sufficiency” and “adequacy” when used in the context of a sample refer to an assessment of whether or not the sample is sufficient as a diagnostic/staging material or other assays, such as for pathological analysis, e.g. whether or not there is enough lymphoid material/cells in the sample for the purposes of subsequent analyses by ROSE, pathology, or other downstream analysis (e.g. next-generation sequencing). Sufficiency can be determined as described in the Examples, and cut-offs or thresholds can be established based on a plurality of samples, the cut-offs or thresholds selected according to a selected or desired specificity and/or sensitivity.

The term “biomarker” as used herein refer to any biomolecule including but not limited to proteins, polypeptides, nucleic acids, lipids, metabolites modifications thereof, that can be used as an indicator of a biological state, in the diagnosis/prognosis of a disease or disorder, and/or in the prediction of the outcome of a treatment or procedure. A biomarker may be used on its own, or in combination with other biomarkers and/or methods. The biomarkers described herein which may be referred to as “lymph node biomarkers” include any biomarker described herein that can be used to determine sufficiency of a sample (e.g., a lymph node sample such as an EBUS-TBNA sample) and can for example be referred to as a sufficiency biomarker, and/or detect metastasis in mediastinal lymph nodes and can for example be referred to as a metastasis biomarker. As used herein, the term metastasis biomarker refers to any of the following: EpCAM, KRT19, SFN, KRT7, INSM1 and/or KRT5, including any subset thereof. As used herein, the term sufficiency biomarker refers to any of the following: CXCL13, CCL21, SIGLEC1 and/or UBD, including any subset thereof. The phrase “one or more lymph node biomarkers” or “one or more biomarkers” can be used to refer to one or more metastasis and/or sufficiency biomarkers.

The term “CXCL13” refers to C-X-C motif chemokine 13 and encompasses variants, isoforms, mutant forms etc., including CXCL13 protein or transcript. For example, the CXCL13 can comprise the sequence as described in UniProt Accession No. 043927.

The term “CCL21” refers to C-C motif chemokine 21 and encompasses variants, isoforms, mutant forms etc., including CCL21 protein or transcript. For example, the CCL21 can comprise the sequence as described in UniProt Accession No. 000585.

The term “SIGLEC1” refers to Sialic acid binding Ig like lectin 1 and encompasses variants, isoforms, mutant forms etc., including SIGLEC1 protein or transcript.

For example, the SIGLEC1 can comprise the sequence as described in UniProt Accession No. Q9BZZ2.

The term “UBD” refers to Ubiquitin D and encompasses variants, isoforms, mutant forms etc., including UBD protein or transcript. For example, the UBD can comprise the sequence as described in UniProt Accession No. 015205.

The term “EpCAM” refers to Epithelial cell adhesion molecule and encompasses variants, isoforms, mutant forms etc., including EpCAM protein or transcript. For example, the EpCAM can comprise the sequence as described in UniProt Accession No. P16422.

The term “KRT19” refers to Keratin, type I cytoskeletal 19 and encompasses variants, isoforms, mutant forms etc., including KRT19 protein or transcript. For example, the KRT19 can comprise the sequence as described in UniProt Accession No. P08727.

The term “SFN” refers to 14-3-3 protein sigma and encompasses variants, isoforms, mutant forms etc., including SFN protein or transcript. For example, the SFN can comprise the sequence as described in UniProt Accession No. P31947.

The term “KRT7” refers to Keratin, type I cytoskeletal 7 and encompasses variants, isoforms, mutant forms etc., including KRT7 protein or transcript. For example, the KRT7 can comprise the sequence as described in UniProt Accession No. P08729.

The term “INSM1” refers to Insulinoma-associated protein 1 and encompasses variants, isoforms, mutant forms etc., including for example INSM1 protein or transcript. For example, the INSM1 can comprise the sequence as described in UniProt Accession No. Q01101.

The term “KRT5” refers to Keratin, type II cytoskeletal 5 and encompasses variants, isoforms, mutant forms etc., including for example KRT5 protein or transcript. For example, the KRT5 can comprise the sequence as described in UniProt Accession No. P13647.

As used herein, the terms “level”, “expression level” and the like when used in the context of a biomarker refers to the amount of the biomarker measured/detected in a sample. The amount of the biomarker can be for example, a concentration, an absolute amount, a relative amount, a normalized amount etc.

The terms “diagnostics”, “diagnosis” and the like refer to determining the presence or absence or severity of a disease, disorder or condition, or determining one or more characteristics of a disease, disorder or condition, including type, grade, and stage.

The term “antibody” as used herein is intended to include monoclonal antibodies including chimeric and humanized monoclonal antibodies, polyclonal antibodies, humanized antibodies, human antibodies, and chimeric antibodies. Single chain antibodies are also contemplated as well as single VH or VL domains or nanobodies. The antibody may be from recombinant sources and/or produced in transgenic animals. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The term also includes antibody binding fragments.

The term “binding fragment” or “antigen binding fragment” as used herein interchangeably is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The term “binding agent” as used herein includes antibodies and binding fragments thereof, monobodies and other synthetic binding proteins, aptamers such as DNA aptamers and RNA aptamers, peptide ligands such as peptidomimetics, natural receptor/ligand or other molecule which selectively binds a selected target (e.g. a sufficiency or metastasis biomarker described herein). Binding agent affinity, such as antibody affinity can be measured for example by ELISA such as indirect ELISA, microscale thermophoresis or Surface Plasmon Resonance (SPR).

The term “point-of-care device” as used herein refers to a device for diagnostics and/or sample assessment that is used at or near the site of patient care. A site of patient care can be, for example, a hospital, a clinic, a physician's office, a patient's home. In the present disclosure, the site of care, for example, can be where EBUS-TBNA is performed (e.g., at bronchoscopy suite).

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The term “consisting of” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 20%, 5-20%, or 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus, for example, a composition containing “a biomarker” includes two or more biomarkers. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be under-stood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Although any methods and materials similar or equivalent to those described herein can also be used in the practice or testing of the present disclosure, examples of methods and materials are now described.

### II. Methods

In one aspect, the present disclosure provides methods for assessment of sufficiency of a lymph node sample, for example for diagnostic assessment. In some embodiments, the lymph node sample is a biopsy. In some embodiments, the lymph node sample is a needle aspirate sample. In some embodiments, the lymph node sample is an EBUS-TBNA sample. A needle rinse of a needle used to obtain a lymph node sample such as an EBUS TBNA-sample, may also be used.

In some embodiments, the methods may comprise measuring the level of one or more, biomarkers, such as one or more sufficiency biomarkers in the sample. In some embodiments, the one or more sufficiency biomarkers are selected from CXCL13 and/or CCL21.

In some embodiments, the one or more sufficiency biomarkers measured in the sample include SIGLEC1 and/or UBD.

For example, the one or more sufficiency biomarkers can be 1 or 2 or more of the sufficiency biomarkers.

In an embodiment, the one or more sufficiency biomarkers is or comprises CXCL13. In an embodiment, one or more sufficiency biomarkers is or comprises CCL21. Other biomarkers can also be assessed. In another embodiment, any one of the lymph node biomarkers can be combined with any one or more of the other lymph node biomarkers.

In an embodiment, the one or more lymph node sufficiency biomarkers are CXCL13 and CCL21.

In an embodiment, the one or more lymph node sufficiency biomarkers further comprises SIGLEC1. In an embodiment, the one or more lymph node sufficiency biomarkers further comprises UBD.

In an embodiment, the one or more lymph node sufficiency biomarkers are CXCL13, CCL21 and SIGLEC1. In an embodiment, the one or more lymph node sufficiency biomarkers are CXCL13, CCL21 and UBD. In an embodiment, the one or more lymph node sufficiency biomarkers are CXCL13, CCL21, SIGLEC1 and UBD.

Where a lymph node sample is determined to be sufficient, the sample can be subject to a downstream application, such as next-generation sequencing, ROSE, and/or a pathological assay and/or assessment.

Where a lymph node sample is determined to be insufficient, one or more subsequent lymph node samples can be obtained and measurement of the level of the one or more lymph node sufficiency biomarkers can be repeated.

The bronchoscope may for example remain positioned to take a subsequent sample while the previous sample, e.g., a first sample or immediately preceding sample, is assessed for sample sufficiency. If the sample is determined to be insufficient, a subsequent sample may be taken. If sufficient, no further sample may be taken.

In an embodiment, the lymph node sample and the one or more subsequent lymph node samples are taken from the same lymph node. In an embodiment, the lymph node sample and the one or more subsequent lymph node samples are taken from different lymph nodes.

In some embodiments, the method further comprises performing an assay to detect metastasis.

In an embodiment, the assay for detecting metastasis comprises measuring the level of one or more lymph node metastasis biomarkers. In some embodiments, the one or more lymph node metastasis biomarkers are selected from EpCAM, KRT19, SFN, KRT7, INSM1 and/or KRT5.

The method can include detecting any combination of the one or more lymph node metastasis biomarkers. In one embodiment, the one or more lymph node metastasis biomarkers are at least 2, at least 3, at least 4, or at least 5 lymph node metastasis biomarkers. In one embodiment, the one or more lymph node metastasis biomarkers are the lymph node metastasis biomarkers.

For example, the one of more lymph node metastasis biomarkers can be 1, 2, 3, 4, 5 or 6 of the lymph node metastasis biomarkers.

In one embodiment, the one or more lymph node metastasis biomarker is or comprises EpCAM. In another embodiment, the one or more lymph node metastasis biomarker is or comprises KRT19. In another embodiment, the one or more lymph node metastasis biomarker is or comprises SFN. In another embodiment, the one or more lymph node metastasis biomarker is or comprises KRT7. In another embodiment, the one or more lymph node metastasis biomarker is or comprises INSM1. In another embodiment, the one or more lymph node metastasis biomarker is or comprises KRT5. In another embodiment, any one of the lymph node metastasis biomarkers can be combined with any one or more of the other lymph node metastasis biomarkers.

The one or more lymph node metastasis biomarkers can be used as diagnostic or prognostic markers for lung cancer, including for example adenocarcinoma (ADC), non-small cell lung cancer (NSCLC), small cell lung carcinoma (SCLC), and squamous lung carcinoma (SQC).

In some embodiments, the one or more lymph node metastasis biomarkers are used as diagnostic and/or prognostic markers for adenocarcinoma (ADC). In an embodiment, the one or more lymph node metastasis biomarkers is or comprises KRT19. In an embodiment, the one or more lymph node metastasis biomarker is or comprises SFN. In an embodiment, the one or more lymph node metastasis biomarker is or comprises KRT7. In an embodiment, the one or more lymph node metastasis biomarker is or comprises INSM1. In an embodiment, the one or more lymph node metastasis biomarker is or comprises KRT5. In another embodiment, any one of the lymph node metastasis biomarkers can be combined with any one or more of the other lymph node metastasis biomarkers.

In some embodiments, the one or more lymph node metastasis biomarkers are used as diagnostic and/or prognostic markers for squamous cell carcinoma (SQC). In an embodiment, the one or more lymph node metastasis biomarkers is or comprises KRT19. In an embodiment, the one or more lymph node metastasis biomarker is or comprises INSM1. In an embodiment, the one or more lymph node metastasis biomarker is or comprises KRT5. In an embodiment, the one or more lymph node metastasis biomarkers is or comprises KRT19 and INSM1. In an embodiment, the one or more lymph node metastasis biomarkers is or comprises KRT19 and KRT5. In an embodiment, the one or more lymph node metastasis biomarkers is or comprises KRT5 and INSM1. In an embodiment, the one or more lymph node metastasis biomarkers is or comprises KRT19, KRT5 and INSM1.

In some embodiments, the one or more lymph node metastasis biomarkers are used as diagnostic and/or prognostic markers for small cell lung carcinoma (SCLC). In an embodiment, the one or more lymph node metastasis biomarkers is or comprises EpCAM. In an embodiment, the one or more lymph node metastasis biomarker is or comprises INSM1. In an embodiment, the one or more lymph node metastasis biomarker is or comprises KRT5. In an embodiment, the one or more lymph node metastasis biomarkers is or comprises EpCAM and INSM1. In an embodiment, the one or more lymph node metastasis biomarkers is or comprises EpCAM and KRT5. In an embodiment, the one or more metastasis lymph node biomarkers is or comprises KRT5 and INSM1. In an embodiment, the one or more lymph node metastasis biomarkers is or comprises EpCAM, KRT5 and INSM1.

The sample used for detecting metastasis can for example be first assessed for sufficiency. In various embodiments, the sample used for detecting metastasis is a sample determined to be sufficient, for example using a method described herein and/or using one or more lymph node sufficiency biomarkers.

Accordingly, in some embodiments, the method for determining sample sufficiency is followed by the method for detecting metastasis.

In some embodiments, the method for determining sample sufficiency and the method for detecting metastasis are performed concurrently.

In some embodiments, the sample is obtained from a subject suspected of having lung cancer. In some embodiments, the sample is obtained from a subject diagnosed as having lung cancer. In some embodiments, the lung cancer is adenocarcinoma, non-small cell lung cancer, small cell lung cancer or squamous cell lung cancer.

In another aspect, the present disclosure provides methods for detecting metastasis, comprising providing a lymph node sample obtained from a subject, measuring a level of one or more lymph node metastasis biomarkers selected from EpCAM, KRT19, SFN, KRT7, INSM1 and/or KRT5 in the sample; wherein the level of the one or more lymph node metastasis biomarkers is indicative of metastasis. The method can include detecting any combination of the one or more lymph node metastasis biomarkers. In one embodiment, the one or more lymph node metastasis biomarkers are at least 2, at least 3, at least 4, or at least 5 lymph node metastasis biomarkers. In one embodiment, the one or more metastasis biomarkers are the lymph node metastasis biomarkers.

For example, the one of more lymph node metastasis biomarkers can be 1, 2, 3, 4, 5 or 6 of the lymph node metastasis biomarkers.

In some embodiments, the lymph node sample is a biopsy. In some embodiments, the lymph node sample is a needle aspirate sample. In some embodiments, the lymph node sample is an EBUS-TBNA sample.

In one embodiment, the one or more lymph node metastasis biomarker is or comprises EpCAM. In another embodiment, the one or more lymph node metastasis biomarker is or comprises KRT19. In another embodiment, the one or more lymph node metastasis biomarker is or comprises SFN. In another embodiment, the one or more lymph node metastasis biomarker is or comprises KRT7. In another embodiment, the one or more lymph node metastasis biomarker is or comprises INSM1. In another embodiment, the one or more lymph node metastasis biomarker is or comprises KRT5. In another embodiment, any one of the lymph node metastasis biomarkers can be combined with any one or more of the other lymph node metastasis biomarkers.

The one or more lymph node metastasis biomarkers can be used as diagnostic or prognostic markers for lung cancer, including for example adenocarcinoma (ADC), non-small cell lung cancer (NSCLC), small cell lung carcinoma (SCLC), and squamous lung carcinoma (SQC).

In some embodiments, the one or more lymph node metastasis biomarkers are used as diagnostic and/or prognostic markers for adenocarcinoma (ADC). In an embodiment, the one or more lymph node metastasis biomarkers is or comprises KRT19. In an embodiment, the one or more lymph node metastasis biomarker is or comprises SFN. In an embodiment, the one or more lymph node metastasis biomarker is or comprises KRT7. In an embodiment, the one or more lymph node metastasis biomarker is or comprises INSM1. In an embodiment, the one or more lymph node metastasis biomarker is or comprises KRT5. In another embodiment, any one of the lymph node metastasis biomarkers can be combined with any one or more of the other lymph node metastasis biomarkers.

In some embodiments, the one or more lymph node metastasis biomarkers are used as diagnostic and/or prognostic markers for squamous cell carcinoma (SQC). In an embodiment, the one or more lymph node metastasis biomarkers is or comprises KRT19. In an embodiment, the one or more lymph node metastasis biomarker is or comprises INSM1. In an embodiment, the one or more lymph node metastasis biomarker is or comprises KRT5. In an embodiment, the one or more lymph node metastasis biomarkers is or comprises KRT19 and INSM1. In an embodiment, the one or more lymph node metastasis biomarkers is or comprises KRT19 and KRT5. In an embodiment, the one or more lymph node metastasis biomarkers is or comprises KRT5 and INSM1. In an embodiment, the one or more lymph node metastasis biomarkers is or comprises KRT19, KRT5 and INSM1.

In some embodiments, the one or more lymph node metastasis biomarkers are used as diagnostic and/or prognostic markers for small cell lung carcinoma (SCLC). In an embodiment, the one or more lymph node metastasis biomarkers is or comprises EpCAM. In an embodiment, the one or more lymph node metastasis biomarker is or comprises INSM1. In an embodiment, the one or more lymph node metastasis biomarker is or comprises KRT5. In an embodiment, the one or more lymph node metastasis biomarkers is or comprises EpCAM and INSM1. In an embodiment, the one or more lymph node metastasis biomarkers is or comprises EpCAM and KRT5. In an embodiment, the one or more metastasis lymph node biomarkers is or comprises KRT5 and INSM1. In an embodiment, the one or more lymph node metastasis biomarkers is or comprises EpCAM, KRT5 and INSM1.

In some embodiments, the level of the one or more lymph node biomarkers is measured by affinity-based assays. Any suitable types of affinity-based assays can be used, including but not limited to immunoassays such as enzyme-linked immunosorbent assays (ELISA), immunohistochemistry (IHC), radioimmunoassays (RIA), fluorescent immunoassays, the practices of which are well known in the art (see, e.g., Ausubel, Frederick M. Current Protocols in Molecular Biology. New York: John Wiley & Sons, 1994, the content of which is incorporated by reference in its entirety).

In an embodiment, the level of the one or more lymph node biomarkers is measured by an enzyme-linked immunosorbent assay (ELISA).

Other assay types that can be used include but not limited to lateral flow assays, mass spectrometry, and electrochemical assays.

In an embodiment, the level of the one or more lymph node biomarkers is measured by a lateral flow assay (LFA). Various types of multiplexed lateral flow assays are contemplated. The detection sites can be spatially separated and be comprised in a single strip. Alternatively several strips can be aligned in an array format. Finally different signal reporters, such as different fluorescent molecules can be utilized that provide different signals.

Affinity-based assays typically comprise the use of one or more binding agents that bind specifically the protein of interest. A variety of binding agents can be used, including but not limited to antibodies and binding fragments thereof, ligands, receptors, monobodies, aptamers, oligonucleotides, and molecularly imprinted polymers. The binding agent may bind the full-length protein or a fragment thereof, an isoform, a pro-protein, a post-translationally modified protein etc.

In some embodiments, the binding agents are antibodies and/or binding fragments thereof.

Binding agents may be produced by any suitable methods known in the art or purchased from commercial sources. Examples of commercially available binding agents that can be used to specifically recognize the lymph node biomarkers disclosed herein include but are not limited to:

| Biomarker | Examples of commercially available binding agents |
|---|---|
| CXCL13 | Thermo Fisher Scientific, CXCL13 antibody (cat no. |
| | PA5-28827) |
| CCL21 | Thermo Fisher Scientific, CCL21 antibody (cat no. |
| | PA5-84055) |
| SIGLEC1 | R&D Systems, Human Siglec-1/CD169 Antibody (cat no. |
| | AF5197) |
| UBD | R&D Systems, Human FAT10 Antibody (cat no. AF8408) |
| EpCAM | Abcam Anti-EpCAM (ab282457) |
| KRT19 | Santa Cruz Anti-cytokeratin 19 (sc-376126) |
| SFN | Abcam anti-14-3-3 sigma (ab86380) |
| KRT7 | Santa Cruz Anti-cytokeratin7 (sc-23876) |
| INSM1 | Santa Cruz (A-8) |
| KRT5 | Santa Cruz Anti-cytokeratin5 sc-32721 |

In general, detecting and/or measuring the level of the one or more lymph node biomarkers by an affinity-based assay involves contacting a lymph node sample with the binding agent.

The binding agent may be directly conjugated to a detectable label or moiety. The binding agent may be detected indirectly, for example, through the use of a secondary binding agent that is conjugated to a detectable label or moiety. A variety of detectable labels and moieties are known in the art, including but not limited to enzymes such as horseradish peroxidase, alkaline phosphatase, β-galactosidase, acetylcholinesterase, and catalase, florescent dyes such as Cy3, Cy5, FITC, radioisotopes such as iodine-125, nanoparticles such as gold nanoparticles.

In some embodiments, the binding agents are conjugated to fluorescent dyes and/or nanoparticles.

In some embodiments, the level of the one or more lymph node biomarkers is measured by transcript levels (e.g., mRNA). Such methods may be amplification-based, for example, qRT-PCR. Transcript levels may be measured by hybridization methods. For example, a nucleic acid probe can be used to capture the mRNA or cDNA of the target biomarker based on complementarity. In some embodiments, nucleic acid probes are used with electrochemical assays to detect and/or measure the level of the one or more lymph node biomarkers.

In some embodiments, the level of the one or more lymph node biomarkers is measured by microarray, for example, GeneChip™ Human Gene 1.0 ST Array (Affymetrix, Santa Clara, CA). For example, the cancer biomarkers EpCAM, KRT19,5,7, SFN and/or INSM1 may be detected by PCR or other nucleic acid method) for example using GeneChip, Human Gene 1.0 ST Array, Affymetrix, Santa Clara, CA.

The methods disclosed herein may be qualitative, semi-quantitative, or quantitative. For example, a qualitative assessment may be used with a lymph node biomarker that is present in sufficient samples but absent in insufficient samples. As used herein, “presence or absence” when used in the context of the methods disclosed herein, means whether or not the lymph node biomarker is present in the sample at a level detectable by the affinity reagent under specific assay conditions. Such a qualitative assessment may be done by the naked eye, as in the detection of the presence or absence of a line on a lateral flow assay device. A semi-quantitative assessment may also be done with a lateral flow assay device, for example, based on the number and the intensity of multiple test lines (see e.g. Parolo C et al., Tutorial: design and fabrication of nanoparticle-based lateral-flow immunoassays. Nat Protoc 15, 3788-3816 (2020), the content of which is incorporated by reference herein in its entirety).

In some embodiments, the level of the one or more lymph node biomarkers is measured using a quantitative immunofluorescence assay, for example using a multiplexing platform.

In some embodiments, the level of the one or more lymph node biomarkers is measured using an electrochemical assay, for example using a microchip sensor as described in Example 3. The method can comprise a probe such as a nucleic acid probe or a peptide nucleic acid probe that specifically binds a biomarker described herein. The probe: biomarker complex can be directly or indirectly bound by a reporter which provides a readout of the level of the biomarker. The reporter, for example, can be an electrochemical reporter, for example a ruthenium based reporter that binds negatively charged phosphate groups or a labelled secondary antibody. For example, electrochemical measurements can be performed using a potentiostat with a three-electrode system featuring a Ag/AgCl reference electrode (BASi), a platinum wire auxiliary electrode, and a biosensing electrode serving as the working electrode. Methods using electrodes that comprise a ruthenium based reporter can include a step of incubating the biosensing electrode in a solution of Ru3+ and scanning using differential pulse voltammetry (DPV).

In some embodiments, the level of the one or more lymph node biomarkers is measured by nucleic acid based methods. Semi-quantitative and quantitative methods for measuring biomarkers based on nucleic acid levels are well known in the art. These methods may comprise amplification of target nucleic acid. A variety of technologies have been developed for fast nucleic acid amplification for molecular diagnostics applications (See e.g. Lee, S. H., Park, S. M., Kim, B. N., Kwon, O. S., Rho, W. Y. and Jun, B. H., 2019. Emerging ultrafast nucleic acid amplification technologies for next-generation molecular diagnostics. Biosensors and Bioelectronics, 141, p.111448, the content of which is incorporated herein in its entirety).

The sample can be any sample that is taken with the expectation of comprising the intended or desired content. For example, the sample can be a biopsy, or an aspirate. As used herein “sample” is intended to refer to a sample that is purported to comprise the desired or intended content, e.g., lymph node tissue or aspirate. The sample can prior to being used in a method described herein, be purified, isolated, diluted (e.g., in saline) or otherwise processed.

The sample can also be a needle rinse. For example, a lymph node sample can be taken by needle aspiration. The needle used can then be rinsed, and the needle rinse may be used as a sample to determine sample sufficiency by the methods disclosed herein.

Any suitable methods to quantify amplified nucleic acid can be used. In some embodiments, the level of the one or more lymph node biomarkers is measured by real time PCR. In some embodiments, the level of the one or more lymph node biomarkers is measured by qRT-PCR. In some embodiments, the level of the one or more lymph node biomarkers is measured by microarray.

Nucleic acid based methods for measuring biomarker levels may not comprise amplification of target nucleic acids. For example, electrochemical assays may be used to detect and/or measure binding of a mRNA biomarker to a complementary nucleic acid probe.

In some embodiments, the level of the one or more lymph node biomarkers is measured by electrochemical detection of hybridization of mRNA or cDNA with nucleic acid probes.

In some embodiments, the method may comprise comparing the level of the one or more lymph node biomarkers with a pre-determined cut-off value, wherein the differential expression is indicative of sample sufficiency. The cut-off value may be determined from a plurality of samples for example EBUS-TBNA samples where sufficiency is confirmed for example, by pathology. The cut-off value may differ depending on the downstream assay(s) to be performed.

Where the sample is a tissue sample, presence or absence may also be assessed by selecting a threshold such as, at least or greater than 5%, at least or greater than 10%, at least or greater than 15% or at least or greater than 20% immunoreactivity, as indication of the biomarker presence. For example, when using immunofluorescence for example, a biomarker can be considered present (e.g., the sample is positive) if a sample is determined to have at least 10% immunoreactivity.

The sample to be assessed can comprise one or more biopsies from a lymph node or one or more biopsies from a plurality of lymph nodes and comprise a pooled sample. The sample can be obtained from any mediastinal lymph node, for example station 4 or 7.

In some embodiments, the method may comprise comparing the level of the one or more lymph node biomarkers with a pre-determined cut-off value, wherein the differential expression is indicative of cancer metastasis. The cut-off value may be determined from a plurality of samples, optionally EBUS-TBNA samples where metastasis is confirmed by pathology.

In some embodiments, a univariate cut-off value is determined for each of the one or more lymph node biomarkers. In some embodiments, more than one of the lymph node biomarkers are combined into a multivariate model with a set of cut-off values of the more than one lymph node biomarkers.

In some embodiments, the multivariate cut-off is about 20 pg of CXCL13 and/or about 1700 pg of CCL21 in an EBUS-TBNA sample. Other values with selected sensitivity and specificity can also be selected. A person skilled in the art would recognize that the cut-off value would depend on any dilution. For example, as shown in the examples, if the lymph node sample is an EBUS TBNA that is diluted in 10 mL of saline, the diluted level would be 2 pg/mL or 170 pg/mL.

In some embodiments, the methods disclosed herein is used in combination with other methods to assess EBUS-TBNA samples. In one embodiment, the methods disclosed herein is used in combination with ROSE.

Using the methods disclosed herein, it may be determined that an EBUS-TBNA sample is sufficient, in which case the bronchoscopist can cease further EBUS-TBNA lymph node sampling.

Using the methods disclosed herein, it may be determined that an EBUS-TBNA sample is not sufficient, in which case additional EBUS-TBNA samples can be collected from the subject, and the methods disclosed herein may be repeated to determine sufficiency of the EBUS-TBNA. These steps may be repeated until a sufficient EBUS-TBNA sample is collected from the subject.

### III. Devices and Kits

An aspect of the present disclosure relates to a device that can be used for measuring the level of one or more lymph node biomarkers disclosed herein.

In some embodiments, the device is a point-of-care (POC) device.

In some embodiments, the device comprises a solid support, comprising a binding agent for CXC13 and/or a binding agent for CCL21.

In some embodiments, the solid support further comprises a binding agent for SIGLEC1 and/or a binding agent for UBD.

In some embodiments, the device is for determining sufficiency of a lymph node sample, optionally using a method described herein.

In some embodiments, the solid support further comprises a binding agent for each of at least 2 of EpCAM, KRT19, SFN, KRT7, INSM1 and KRT5. In some embodiments, the solid support further comprises a binding agent for each of at least 3 of EpCAM, KRT19, SFN, KRT7, INSM1 and KRT5. In some embodiments, the solid support further comprises a binding agent for each of at least 4 of EpCAM, KRT19, SFN, KRT7, INSM1 and KRT5. In some embodiments, the solid support further comprises a binding agent for each of at least 5 of EpCAM, KRT19, SFN, KRT7, INSM1 and KRT5.

In some embodiments, the device is for determining sufficiency and for detecting metastasis in a lymph node sample, optionally using a method described herein.

In some embodiments, the device comprises a test panel of binding agents. In an embodiment, the test panel of binding agents is or comprises a binding agent for CXC13 and/or a binding agent for CCL21. In an embodiment, the test panel of binding agent is or comprises a binding agent for CXC13, a binding agent for CCL21 and/or a binding agent for SIGLEC1. In an embodiment, the test panel of binding agent is or comprises a binding agent for CXC13, a binding agent for CCL21 and/or a binding agent for UBD. In an embodiment, the test panel of binding agent is or comprises a binding agent for CXC13, a binding agent for CCL21, a binding agent for SIGLEC1 and/or a binding agent for UBD.

In an embodiment, the test panel of binding agents further comprises a binding agent for each of at least 2 of EpCAM, KRT19, SFN, KRT7, INSM1 and KRT5. In an embodiment, the test panel of binding agents further comprises a binding agent for each of at least 3 of EpCAM, KRT19, SFN, KRT7, INSM1 and KRT5. In an embodiment, the test panel of binding agents further comprises a binding agent for each of at least 4 of EpCAM, KRT19, SFN, KRT7, INSM1 and KRT5. In an embodiment, the test panel of binding agents further comprises a binding agent for each of at least 5 of EpCAM, KRT19, SFN, KRT7, INSM1 and KRT5.

In some embodiments, the device comprises a solid support, comprising a binding agent for at least 2 of EpCAM, KRT19, SFN, KRT7, INSM1 and KRT5. In some embodiments, the device comprises a solid support, comprising a binding agent for at least 3 of EpCAM, KRT19, SFN, KRT7, INSM1 and KRT5. In some embodiments, the device comprises a solid support, comprising a binding agent for at least 4 of EpCAM, KRT19, SFN, KRT7, INSM1 and KRT5. In some embodiments, the device comprises a solid support, comprising a binding agent for at least 5 of EpCAM, KRT19, SFN, KRT7, INSM1 and KRT5.

In some embodiments, the device comprises a test panel of binding agents. In an embodiment, the test panel of binding agents is or comprises a binding agent for each of at least 2 of EpCAM, KRT19, SFN, KRT7, INSM1 and KRT5. In an embodiment, the test panel of binding agents is or comprises a binding agent for each of at least 3 of EpCAM, KRT19, SFN, KRT7, INSM1 and KRT5. In an embodiment, the test panel of binding agents is or comprises a binding agent for each of at least 4 of EpCAM, KRT19, SFN, KRT7, INSM1 and KRT5. In an embodiment, the test panel of binding agents is or comprises a binding agent for each of at least 5 of EpCAM, KRT19, SFN, KRT7, INSM1 and KRT5.

In some embodiments, the device is for detecting metastasis in a lymph node sample, optionally using a method described herein.

In some embodiments, the binding agents are antibodies and/or binding fragments thereof. In some embodiments, the binding agents are conjugated to a detectable label such as fluorescent dyes and/or nanoparticles.

In some embodiments, the device is for use with a method disclosed herein.

Another aspect of the present disclosure relates to point-of-care devices and kits for use with the methods disclosed herein. In some embodiments, the kits and devices are for use in the determination of sample sufficiency and/or detection of metastasis in a lymph node sample. In some embodiments, the kits and devices can comprise one or more affinity reagents that specifically bind to one or more of the lymph node biomarkers disclosed herein.

In some embodiments, the kit comprises ELISA. ELISA is a well-established platform for detecting and/or measuring the level of a biomarker. Antibodies are typically used as the binding agent, but any suitable binding agents can be used, such as aptamers, molecularly imprinted polymers, ligands, and receptors.

It is also well established that ELISA can be performed in a variety of formats, including but not limited to sandwich ELISA and competitive ELISA. Any suitable ELISA format can be used with the kit disclosed herein. In some embodiments, the kit can comprise one or more binding agents that recognizes the one or more lymph node biomarkers disclosed herein, one or more secondary enzyme-linked antibodies, and reagents for blocking, washing and colour development.

In some embodiments, the kit can comprise a lateral flow assay. Lateral flow assay is a well-established point-of-care diagnostic platform. Different formats of LFA are available, for example, sandwich LFA and competitive LFA. A LFA may detect one biomarker, or it may detect more than one biomarker if a multiplex format is used. For example, different fluorescent labels may be used for different antibodies on the same device to allow multiplexing. Other labels that can be used include but not limited to nanoparticles such as gold nanoparticles and carbon nanoparticles, enzymes, etc. Any suitable affinity agent may be used, including but not limited to antibodies and fragments thereof, aptamers, ligands, receptors, oligonucleotides, and molecularly imprinted polymers (See e.g. Sajid, M., Kawde, A. N. and Daud, M., 2015. Designs, formats and applications of lateral flow assay: A literature review. Journal of Saudi Chemical Society, 19 (6), pp. 689-705, the content of which is incorporated herein in its entirety).

A LFA may be a qualitative, semi-quantitative and/or quantitative measurement of a biomarker. A qualitative LFA typically refers to detecting the presence or absence of the test line. When used in the context of the methods disclosed herein, “presence or absence” means whether or not the biomarker is present in the lymph node sample at a level detectable by under the specific assay conditions. A semi-quantitative LFA typically involves semi-quantitative evaluation of the intensity of the test line by visual inspection. Quantitative LFA typically involves use of a detection system, e.g. a fluorescence reader if a fluorescent label is used. Sensitivity of a LFA is affected by various factors such as pore size of the nitrocellulose membrane and flow rate. A person skilled in the art would understand that a LFA for use with the methods disclosed herein can be optimized based on those factors affecting sensitivity (See e.g. Bahadir E B & Sezgintürk M K (2016) Lateral flow assays: Principles, designs and labels. TrAC Trends in Analytical Chemistry, 82, 286-306, the content of which is incorporated herein in its entirety).

A lateral flow assay device typically consists of a small plastic cassette that houses a reaction pad separated into designated zones. The patient sample is loaded through a small opening in the cassette and rapidly flows over the ‘conjugate’ zone where detection antibodies are pre-loaded within a protective matrix. Samples then flow to the ‘reaction’ zone containing immobilized capture antibodies and an optical window. At the terminus is the ‘wicking zone’, which acts as a pump and waste reservoir that drives flow through the device via passive capillary forces. For ultrasensitive quantitative protein analysis, detection antibodies are fluorescently labelled and the LFD requires the use of a companion cassette reader.

Accordingly, in an embodiment, the device comprises:
(a) a sample pad for loading a sample on the device;
(b) a conjugate pad, comprising binding agents for each of the one or more lymph node metastasis biomarkers;
(c) a reaction zone for capturing lymph node metastasis biomarkers conjugated to binding agents;
(d) a control zone, comprising a positive control; and
(e) a wicking zone for driving flow of the sample through the device;
wherein the sample after being loaded onto the sample pad flows through (i) the conjugate pad, (ii) the reaction zone, and (iii) the control zone into the wicking zone.

A lateral flow assay device can enable rapid feedback on lymph node sample sufficiency and downstream pathological testing.

Accordingly, the point-of-care device of the present disclosure is or comprises a lateral flow assay for measuring the level of one or more lymph node sufficiency biomarkers selected from CXCL13 and/or CCL21, and optionally SIGLEC1 and/or UBD, for determining sample sufficiency.

In some embodiments, the lateral flow assay is further for measuring the level of one or more lymph node metastasis biomarkers including EpCAM, KRT19, SFN, KRT7, INSM1 and/or KRT5 for detecting metastasis. For example, the binding agents for both the one or more lymph node sufficiency biomarkers and the one or more lymph node metastasis biomarkers can be present on the same lateral flow assay device, allowing for example interrogation of both sample sufficiency and metastasis status in a single assay.

In some embodiments, the lateral flow assay device of the present disclosure comprises a sample pad for loading a sample onto the device; a conjugate pad, comprising binding agents for each of the one or more lymph node biomarkers; a reaction zone for capturing lymph node biomarkers conjugated to binding agents; a control zone, comprising a positive control; and a wicking zone for driving flow of the sample through the device; wherein the sample after being loaded onto the sample pad flows through (i) the conjugate pad, (ii) the reaction zone, and (iii) the control zone into the wicking zone.

In some embodiments, the binding agents are fluorescently labelled. In some embodiments, the binding agents are conjugated to nanoparticles.

Nucleic acid-based assays may be used with the methods disclosed herein.

In some embodiments, the kit can comprise one or more reagents for a PCR assay. In an embodiment, the PCR assay comprises a qRT-PCR assay. In an embodiment, the PCR assay comprises microarray. The kit can comprise reagents such as reverse transcriptase, master mixes, primers, oligonucleotides, labels and/or other components such as microarray chips, purification columns etc.

In some embodiments, the kit can comprise an electrochemical assay. In electrochemical assays, a variety of recognition elements can be employed, for example, antibodies and binding fragments thereof, binding proteins (e.g. receptors, ligands), enzymes, aptamers, nucleic acids, and MIFs. In some embodiments, the recognition element can be affinity-based. In some embodiments, the affinity-based recognition element can be coupled to a label to generate an electroactive species (See e.g., Labib, M., Sargent, E. H. and Kelley, S. O., 2016. Electrochemical methods for the analysis of clinically relevant biomolecules. Chemical reviews, 116 (16), pp. 9001-9090; Grieshaber, D., Mackenzie, R., Vörös, J. and Reimhult, E., 2008. Electrochemical biosensors-sensor principles and architectures. Sensors, 8 (3), pp. 1400-1458; the contents of which are incorporated herein in their entireties).

Any suitable electrochemical transducer modes can be used, for example, amperometirc/voltametric, potentiometric, conductometric, impedimetric etc. For example, the recognition element may generate an electroactive species that can be oxidized or reduced to produce ions, which can be measured by potentiometric techniques.

Electrochemical assays may be used with different device types, such as chip-based devices and microfluidic devices (See e.g. Labib, M., Sargent, E. H. and Kelley, S. O., 2016. Electrochemical methods for the analysis of clinically relevant biomolecules. Chemical reviews, 116 (16), pp. 9001-9090).

In some embodiments, the method or kit comprises a microchip-based sensor or sensors of the one or more lymph node biomarkers. In some embodiments, the microchip-based sensor comprises nucleic acid probes designed to hybridize with the mRNA (or cDNA) of the one or more lymph node biomarkers.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

### EXAMPLES

### Example 1

### Lymph Node Biomarkers to Detect Sample Sufficiency

### Methods

### Patient Population

Patients undergoing mediastinal lymph node staging with ROSE at Toronto General Hospital were prospectively enrolled in this study from March to September 2021. Clinical data, including demographics, EBUS-TBNA procedure reports, ROSE and final pathologic diagnosis, were collected.

### Biomarker Testing

Blood samples from the pulmonary artery and whole lymph nodes were recovered from resected lungs. RNA was purified from tissue or white blood cells (Qiagen) and used for cDNA synthesis (Wisent BioProducts). Quantification of target genes (CXC13, CCL21, UBD, CD20, POU2AF1, CCL19, SIGLEC1, CD19, CD21, and CXCL9) was performed using quantitative polymerase chain reaction (qPCR) (Bio-Rad) normalized with a housekeeping gene, GAPDH.

A 200 μL aliquot of the 10 mL saline needle rinse solution was collected from the EBUS-TBNA procedure. Cytokine concentrations for CXCL13 (R&D Systems) and CCL21 (OriGene Technologies) were determined by quantitative immunofluorescence.

### Statistical Analysis

Kruskal-Wallis and Mann-Whitney U tests were used to analyze biomarker levels. Multiple comparisons were adjusted using Dunn's correction. The area under the receiver operating characteristic (AUROC) curve was used to assess the performance of the lymphoid markers to predict sample adequacy, with the null hypothesis that performance was 50%. For the assessment of sample sufficiency determination of biomarkers vs ROSE, specimens with low cellularity and limited evaluation by pathology were considered as insufficient samples. For CXCL13 concentrations below the lower limit of detection (1.6 pg/mL), samples were assigned a value of 0.8 pg/mL for analysis. All analyses were conducted using GraphPad Software.

### Results

A total of 49 EBUS-TBNA samples were collected from 14 patients in this study (Table 1). ROSE indicated that 80% of samples contained sufficient lymphoid material for diagnostic testing (Table 1). Final pathologic evaluation revealed that 17 (35%) samples were diagnosed as malignant compared to 19 (39%) that were normal. Notably, of the normal diagnoses, the final pathologic evaluation was limited due to low cellularity in 9 (18%) EBUS-TBNA samples.

TABLE 1
Study Population Characteristics

| | All | |
|---|---|---|
| | Patients | |
| Number of Patients | 14 | |
| Male (%) | 10 | (71%) |
| Median Age [SD] - Years | 70 | [10] |
| Number of EBUS-TBNA | 49 | |
| Samples | | |
| Median Number of Passes [IQR] | 3 | [2-4] |
| Lymph Node Station | | |
| 4R (%) | 15 | (31%) |
| 4L (%) | 12 | (24%) |
| 7 (%) | 15 | (31%) |
| 10L (%) | 1 | (2%) |
| 11R (%) | 2 | (4%) |
| 11L (%) | 4 | (8%) |
| ROSE Result | | |
| Sufficient (%) | 39 | (80%) |
| Insufficient (%) | 10 | (20%) |
| Final Pathology Result | | |
| Positive-Adenocarcinoma (%) | 9 | (18%) |
| Positive-NSCLC (%) | 2 | (4%) |
| Positive-SCLC (%) | 4 | (8%) |
| Positive-Squamous Cell (%) | 1 | (2%) |
| Positive-Necrotic (%) | 1 | (2%) |
| Negative (%) | 10 | (20%) |
| Negative-Low Cellularity (%) | 9 | (18%) |
| Insufficient Sample (%) | 5 | (10%) |
| No Report (%) | 8 | (16%) |
| Biomarker Levels | | |
| CXCL13 pg/mL (Median [IQR]) | 9 | [0.8-36] |
| CCL21 pg/mL (Median [IQR]) | 398 | [253-549] |

Legend: SD = standard deviation; IQR = interquartile range; EBUS-TBNA = endobronchial ultrasound-guided transbronchial needle aspiration; NSCLC = non-small cell lung cancer; SCLC = small cell lung cancer; CXCL13 = C-X-C motif chemokine ligand 13; CCL21 = C-C motif chemokine ligand 21.

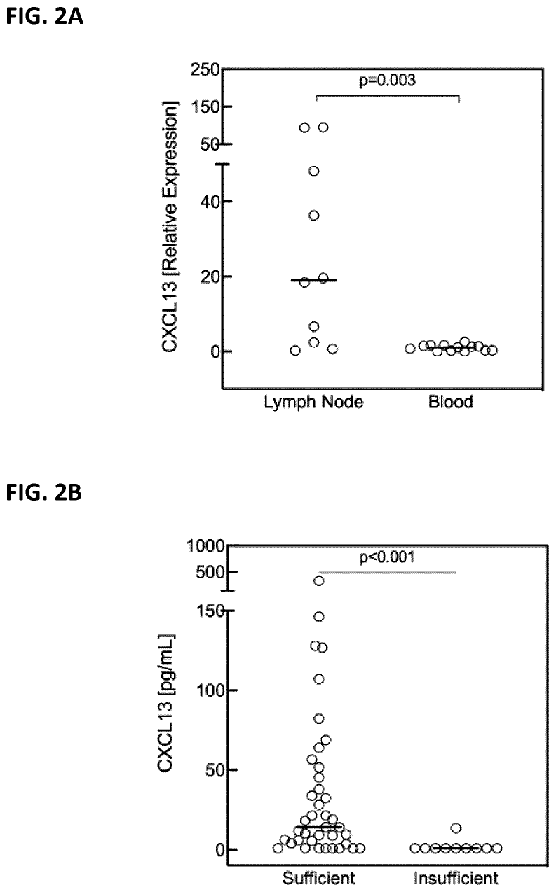

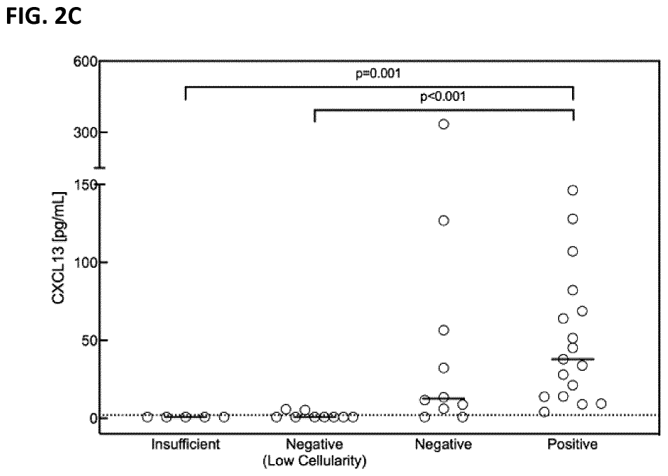

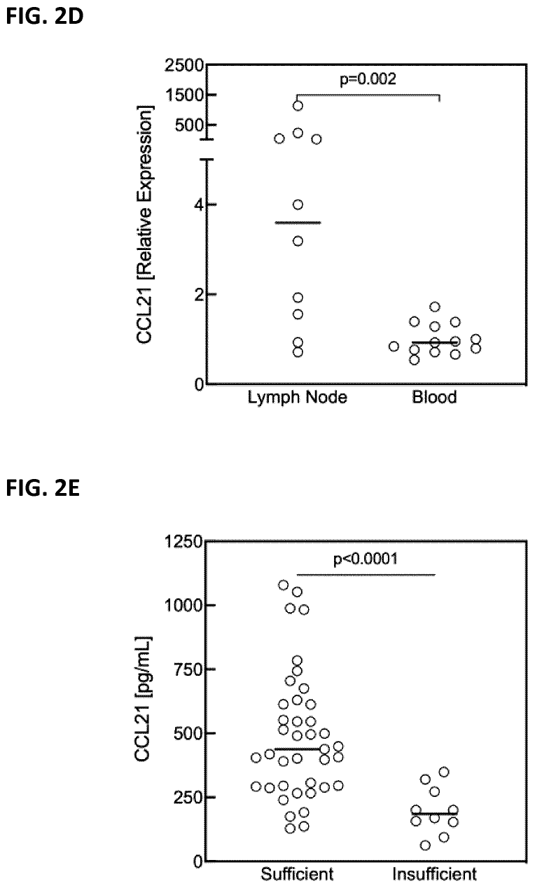

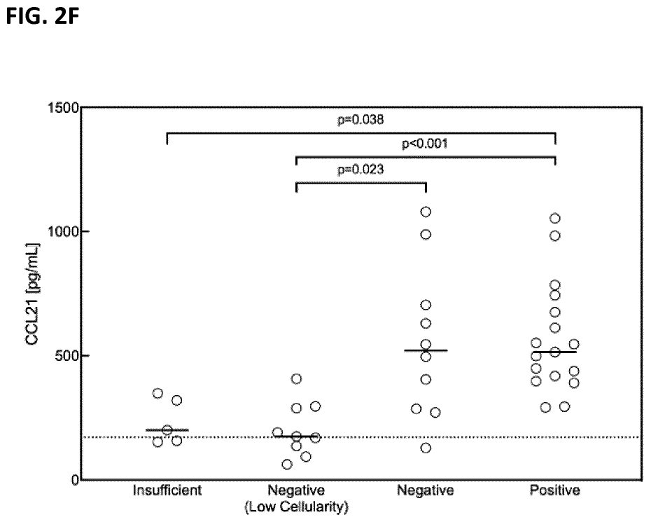

Using the Human Protein Atlas⁹ and the BioGPS,¹⁰ an initial set of ten potential lymphoid biomarkers that were likely to have significantly higher expression levels in lymph node tissue versus whole blood was selected in silico. qPCR showed no difference in the median expression in lymph nodes (n=10) versus blood samples (n=13) for six biomarker candidates (CD20, POU2AF1, CCL19, CD19, CD21, and CXCL9); SIGLEC1 and UBD levels were increased 2- and 3-fold respectively. CCL21 and CXCL13 showed the highest fold increase at 4- and 19-fold increased expression in lymph node tissue (FIGS. 2A, 2D). These two biomarkers in the EBUS-TBNA samples (n=49) collected in the bronchoscopy suite were then validated. Both CXCL13 and CCL21 protein levels were significantly elevated in EBUS-TBNA samples, with sufficient diagnostic material as judged by ROSE (FIGS. 2B, 2E). As univariate predictors of sufficient sample, CXCL13 and CCL21 biomarkers performed well, with an AUROC of 86% [95% CI: 75-97%, p<0.001] and 88% [95% CI: 78-98%, p<0.001] respectively.

Next, the performance of CXCL13 and CCL21 was compared with the final pathologic evaluation performed on the EBUS-TBNA samples. Specimens with yields that were insufficient for or had low cellularity and limited final pathologic evaluation showed very low levels of CXCL13 and CCL21 (FIGS. 2C and 2F). In samples with higher diagnostic yields, CXCL13 and CCL21 levels were elevated regardless of malignancy status (FIGS. 2C and 2F)). Lastly, the lymph node biomarkers were combined into a simple multivariate model with an optimal cut-off of 2 pg/mL and 170 pg/mL for CXCL13 and CCL21 to arrive at a biomarker-based determination of sample sufficiency and evaluated predictive performance alongside ROSE (Table 2). ROSE and the biomarker-based approach showed equivalent sensitivity 96% [95% CI: 81-100%] for predicting samples with sufficient diagnostic material; however, the biomarker-based approach had a considerably better specificity (86% [95% CI: 57-98%)] compared to ROSE (57% [95% CI: 29-82%]).

TABLE 2
Predictive Performance of ROSE and lymph node
Biomarkers for Sample Adequacy using CXCL13
(2 pg/mL) and CCL21 (170 pg/mL) cutoffs

| | |
|---|---|
| ROSE | CXCL13 & CCL21 |

| | | | | |
|---|---|---|---|---|
| Sensitivity [95% CI] | 96% | [81-100%] | 96% | [81-100%] |
| Specificity [95% CI] | 57% | [29-82%] | 86% | [57-98%] |
| PPV [95% CI] | 81% | [70-89%] | 93% | [78-98%] |
| NPV [95% CI] | 89% | [53-98%] | 92% | [63-99%] |
| Accuracy [95% CI] | 83% | [68-93%] | 93% | [80-98%] |

Legend:
CI = confidence interval;
ROSE = rapid onsite evaluation;
CXCL13 = C-X-C motif

### Discussion

Measuring CXCL13 and CCL21 protein levels in EBUS-TBNA samples performs equally well as ROSE in the determination of sufficient yield for diagnostic purposes. Importantly, the biomarker-based approach can better detect samples with low cellularity, which is extremely valuable for novel downstream testing approaches such as next generation sequencing for lung cancer.¹¹

The multivariate biomarker approach accurately reflects EBUS-TBNA samples arising from predominantly lymphoid tissue and, therefore, suitable for subsequent diagnostic testing.

### Example 2

### Lymph Node Biomarkers for the Detection of Metastasis in Mediastinal Lymph Nodes

### Methods

### Clinical Samples:

EBUS-TBNA sufficient (adequate) and insufficient (inadequate) samples (n=20) were obtained by EBUS-TBNA at UHN (Toronto, ON) and placed in 10 mL of sterile saline solution. 200 μL was retained and snap-frozen for protein testing and the remaining sample was sent for pathology analysis.

n=24 lung cancer metastatic lymph nodes (10 adenocarcinomas, 6 squamous cell carcinomas, 2 large cell carcinomas, and 6 small cell carcinomas) and 10 normal lymph nodes were obtained by EBUS-TBNA in UHN (Toronto, ON).

Tissue sample of 79 adenocarcinomas (ADCs), 55 squamous cell carcinomas (SQCs), 55 small cell lung carcinomas (SCLCs) and 133 normal lymph nodes (NL), resected at the Department of Thoracic Surgery of Kumamoto University Hospital (Kumamoto, Japan), were obtained from 189 patients.

### Cell Lines:

Five ADC cell lines (A549, H1975, H2009, H358, and H4006), four SQC cell lines (H2170, H226, H2066, and H520) and five SCLC cell lines (H69, H82, H889, H526, and H69AR) were purchased from ATCC (Manassas, VA).

### IHC Staining:

Formalin-fixed, paraffin-embedded specimens were cut into sections (4 μm thick) and mounted onto MAS-GP-coated slides. After being deparaffinized and rehydrated, the sections were heated using an autoclave in 0.01 mol/L citrate buffer (pH 7.0) for antigen retrieval. The sections were incubated with 0.3% H2O2 in absolute methanol for 20 minutes to block endogenous peroxidase activity. Then, the sections were incubated with Protein Block Serum Free Reagent (Dako, Glostrup, Denmark) for 30 minutes to block non-specific staining. After this blocking step, the sections were incubated with the primary antibodies at 4° C. overnight. This was followed by sequential 1-hour incubations with the secondary antibodies and visualized with the Liquid DAB+ Substrate Chromogen System. All slides were counterstained with hematoxylin for 30 seconds before being dehydrated and mounted. The specificity of immunolabeling of each antibody was tested by using normal mouse IgG and normal rabbit IgG, and no staining was observed.

### Electrochemical Assay:

Electrochemical measurements were performed on a Bioanalytical Systems (BASi) epsilon potentiostat with a three-electrode system featuring a Ag/AgCl reference electrode (BASi), a platinum wire auxiliary electrode, and the biosensing electrode serving as the working electrode. Electrodes were incubated in a solution of Ru3+ then scanned using differential pulse voltammetry (DPV).

### Statistical Analysis:

Statistical calculations and analysis were carried out using Prism 7 (GraphPad) and SPS (IBM) software. For all statistical calculations, a p-value of less than 0.05 was considered statistically significant.

### Results

qRT-PCR analysis of to examine gene expression in metastatic vs. non-metastatic EBUS-TBNA lymph node (LN) samples.

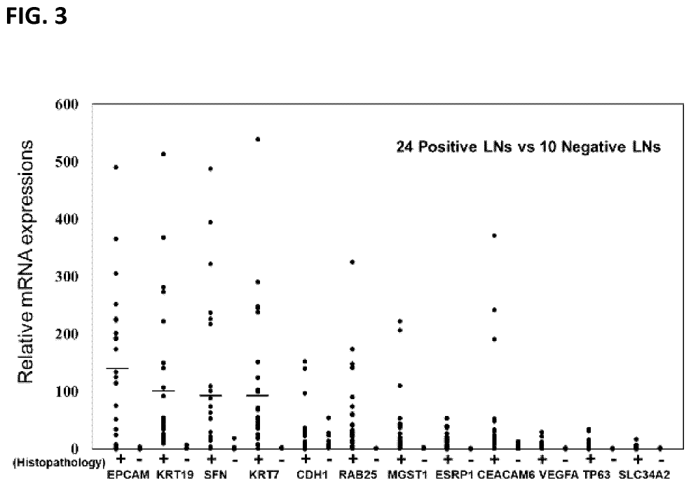

The expression of the lymph node biomarkers (EpCAM, KRT19, SFN, KRT7, CDH1, RAB25, MGST1, ESRP1, CEACAM6, VEGFA, TP63, and SLC34A2) was examined by qRT-PCR. As shown in FIG. 3, the expression of four genes (EpCAM, KRT19, SFN and KRT7) was higher in histopathology confirmed lung cancer metastatic lymph (positive (+)) nodes than those expression in normal lymph nodes (negative (−)).

### Expression of EpCAM, KRT19, SFN, and KRT7 in Lung Cancer Cell Lines

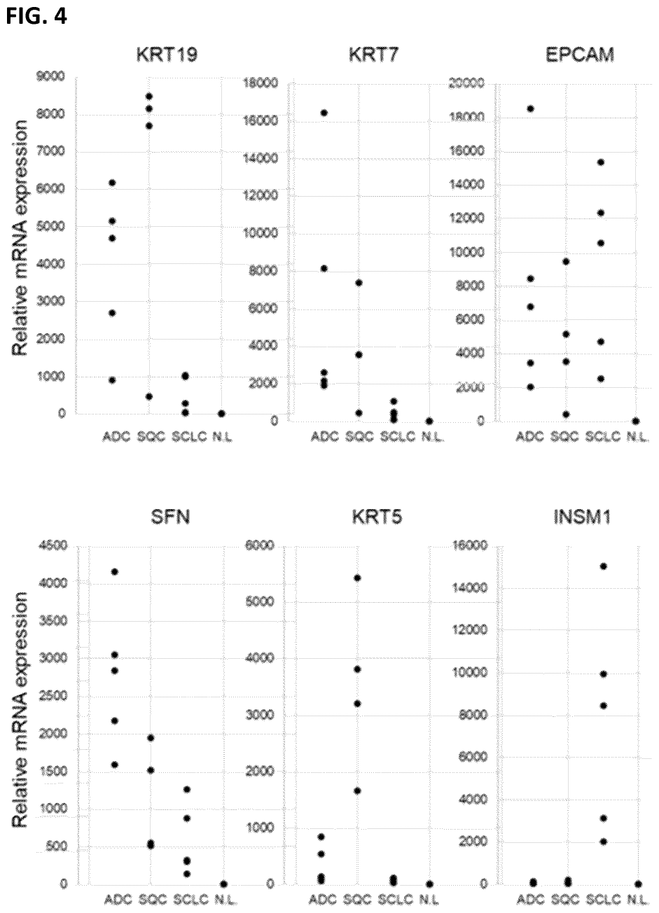

FIG. 4 shows that ADC cell lines had high expression of all EBUS-TBNA genes. SQC cell lines had high expression of KRT19. SCLC had high expression of EpCAM. Two additional genes (INSM1 and KRT5) were used as pathological diagnostic markers for the detection of SCLC and SQC lung cancers. KRT5 was expressed strongly in SQC cell lines, and INSM1 was expressed in SCLC cell lines.

### Immunohistochemical Staining of Lymph Node Biomarkers in Surgically Resected Lymph Node Samples

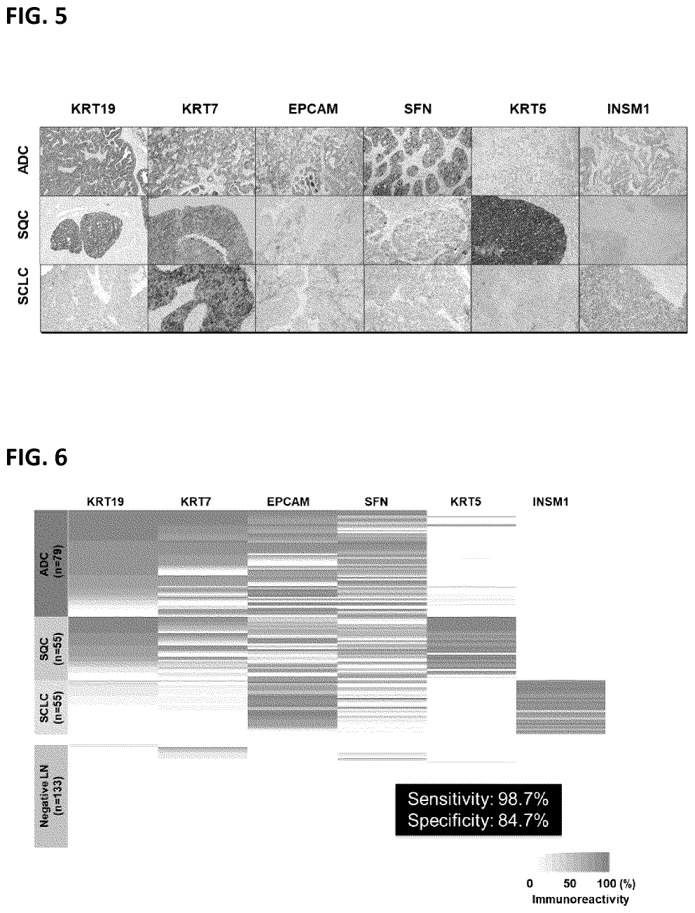

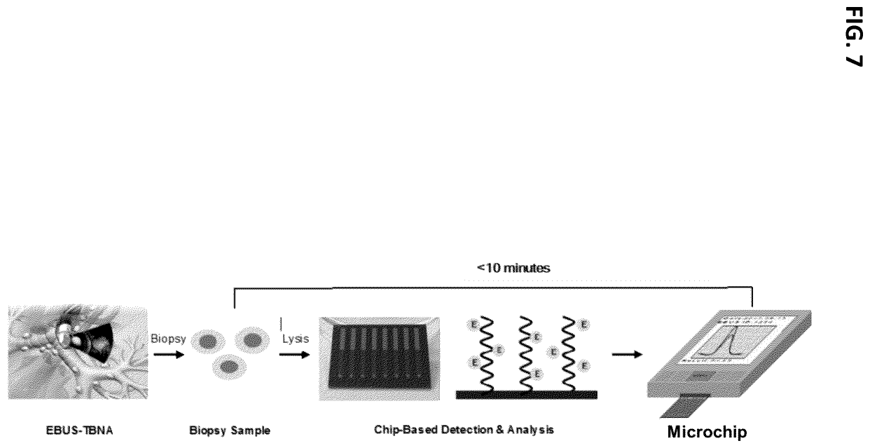

FIG. 5 shows representative IHC images of lymph node biomarkers in ADC, SQC, and SCLC lymph node samples. The immunoreactivity of each lymph node biomarker is summarized for all samples (n=79 ADCs, n=55 SQCs, n=55 SCLCs and n=133 normal lymph nodes (NL)) in FIG. 6. Samples with more than 10% immunoreactivity were considered as positive cases for the respective cancer cell type. Sensitivity and specificity calculations are summarized in Table 3.

KRT19 was positive in 75 ADCs, in 51 SQCs, in 14 SCLCs, and in 4 N.L. (sensitivity 74.4%, specificity 96.9%). KRT7 was positive in 74 ADCs, in 48 SQCs, in 14 SCLCs, and in 13 N.L. (sensitivity 72.8%, specificity 97.7%) EPCAM was positive in 74 ADCs, in 50 SQCs, in 53 SCLCs, and in 0 N.L. (sensitivity 80.9%, specificity 100%). SFN was positive in 73 ADCs, and in 47 SQCs, in 19 SCLCs, and in 9 N.L. (sensitivity 73.5%, specificity 67.6%). KRT5 was expressed in 12 ADCs, in 52 SQCs, in 0 in SCLCs, and in 1 N.L. (sensitivity 29.1%, specificity 98.4%). INSM1 was positive in 0 ADCs, in 3 SQCs, in 55 SCLCs, and in 2 N.L. (sensitivity 29.3%, specificity 98.4%).

TABLE 3
Summary of the diagnostic accuracy of lymph node
biomarkers to detect metastatic lung cancer

| | KRT19 | KRT7 | EpCAM | SFN | KRT5 | INSM1 |
|---|---|---|---|---|---|---|
| Sensitivity | 74.4 | 72.8 | 80.9 | 73.5 | 29.1 | 29.3 |
| (%) | | | | | | |
| Specificity | 96.9 | 97.7 | 100 | 67.6 | 98.4 | 98.4 |
| (%) | | | | | | |

### Example 3

### Point-of-Care Device for Rapid Detection of Lymph Node Biomarkers

### Rapid Detection of Lymph Node Biomarkers

Rapid detection of lymph node biomarkers can be enabled by point-of-care technologies. Microchip-based sensors are an example of technologies that enable the rapid analysis of gene expression in EBUS-TBNA samples. The microchips are fabricated using a silicon wafer as a base substrate with gold electrodes are patterned on the microchips using standard photolithographic processes. A small circular aperture is exposed at the end of each electrode which represents the biosensing portion of the microchips. Complementary probes designed to detect the genes of interest are composed of peptide nucleic acid, a synthetic analogue of DNA. Each probe is chemically modified with a thiol group that allows for the biofunctionalization of the gold aperture of each electrode on the microchip. The functionalized microchips are then exposed to the EBUS-TBNA samples, where hybridization is allowed to occur for <10 minutes. EBUS-TBNA samples or other lymph node transcript samples can be used with or without purification of mRNA prior to hybridization. Following washing, each microchip is exposed to an electrochemical reporter—a ruthenium-based chemical compound—which binds to the negatively charged phosphate backbone of the bound nucleic acid. Using a standard potentiostat, an electrochemical scan is performed which measures the amount of ruthenium present on the microchip and thereby indicates bound nucleic acids and, as such, the presence of the EBUS-TBNA genes of interest.

### Rapid Detection of Lymph Node Biomarkers Performance Characteristics

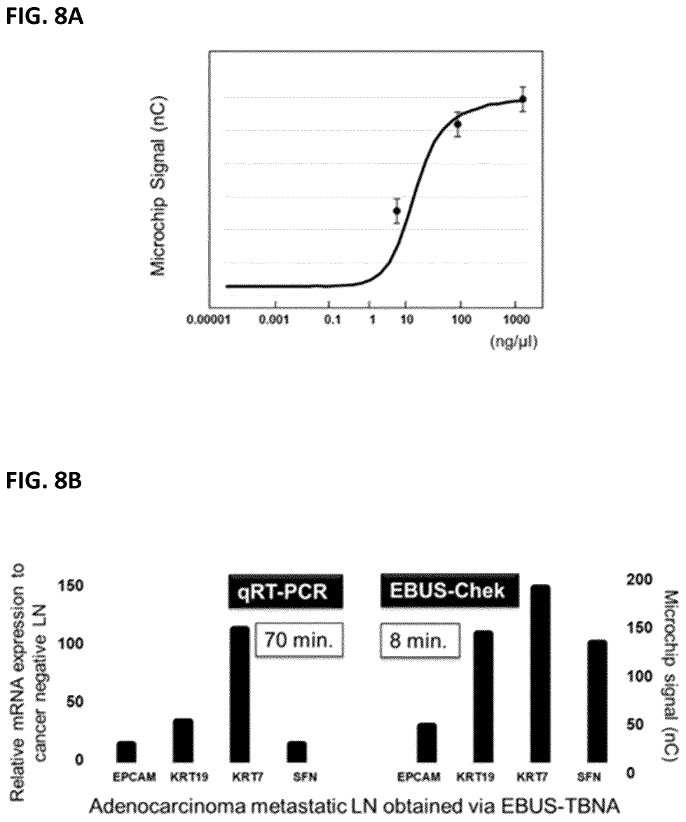

Using the microchip-based approach, EpCAM mRNA was detected and produced electrical currents that were proportional to the amount of nucleic acid present in EBUS-TBNA samples (FIG. 8A). When compared to conventional PCR, the fast hybridization approach enabled by EBUS-TBNA microchips produced similar lymph node biomarker results in adenocarcinoma samples (FIG. 8B).

### CITATIONS FOR REFERENCES REFERRED TO IN THE SPECIFICATION

1. Canadian Cancer Society's Advisory Committee on Cancer Statistics. Canadian Cancer Statistics 2017. Toronto, ON: Canadian Cancer Society.
2. Siegel R, Naishadham D, & Jemal A. Cancer statistics 2012. CA: A Cancer Journal for Clinicians 2012, 62:10-29, doi: 10.3322/caac.20138.
3. Yasufuku K, Chiyo M, Sekine Y, et al. Real-time endobronchial ultrasound-guided transbronchial needle aspiration of mediastinal and hilar lymph nodes. Chest. 2004; 126 (1): 122-128.
4. Silvestri G A, Gonzalez A V, Jantz M A, et al. Methods for staging non-small cell lung cancer: Diagnosis and management of lung cancer, 3rd ed: American College of Chest Physicians evidence-based clinical practice guidelines. Chest. 2013; 143 (5 Suppl): e211S-e250S.
5. Cameron, S. E., Andrade, R. S. & Pambuccian, S. E. Endobronchial ultrasound-guided transbronchial needle aspiration cytology: a state of the art review. Cytopathology: official journal of the British Society for Clinical Cytology 21, 6-26, doi: 10.1111/j. 1365-2303.2009.00722.x (2010).
6. Navani, N. et al. Endobronchial ultrasound-guided transbronchial needle aspiration prevents mediastinoscopies in the diagnosis of isolated mediastinal lymphadenopathy: a prospective trial. American journal of respiratory and critical care medicine 186, 255-260, doi: 10.1164/rccm.201203-0393OC (2012).
7. Oki M, Saka H, Kitagawa C, et al. Rapid on-site cytologic evaluation during endobronchial ultrasound-guided transbronchial needle aspiration for diagnosing lung cancer: a randomized study. Respiration. 2013; 85 (6): 486-492.
8. Trisolini R, Cancellieri A, Tinelli C, et al. Randomized Trial of Endobronchial Ultrasound-Guided Transbronchial Needle Aspiration With and Without Rapid On-site Evaluation for Lung Cancer Genotyping. Chest. 2015; 148 (6): 1430-1437.
9. Uhlén M, Fagerberg L, Hallström B M, et al. Proteomics. Tissue-based map of the human proteome. Science. 2015; 347 (6220): 1260419.
10. Wu C, Orozco C, Boyer J, et al. BioGPS: an extensible and customizable portal for querying and organizing gene annotation resources. Genome Biol. 2009; 10 (11): R130.
11. Labarca G, Folch E, Jantz M, Mehta H J, Majid A, Fernandez-Bussy S. Adequacy of Samples Obtained by Endobronchial Ultrasound with Transbronchial Needle Aspiration for Molecular Analysis in Patients with Non-Small Cell Lung Cancer. Systematic Review and Meta-Analysis. Ann Am Thorac Soc. 2018; 15 (10): 1205-1216.

1. A method of determining lymph node sample sufficiency, the method comprising: (a) providing a lymph node sample obtained from a subject; and (b) measuring a level of one or more lymph node sufficiency biomarkers selected from CXCL13 and/or CCL21 in the sample; wherein the level of the one or more lymph node sufficiency biomarkers is indicative of sample sufficiency.

2. The method of claim 1, further comprising comparing the level of the one or more lymph node sufficiency biomarkers to a pre-determined cut-off value or set of predetermined cut-off values.

3. The method of claim 2, wherein the predetermined cut-off value is determined from a plurality of sufficient samples.

4. The method of any of the preceding claims, further comprising providing a subsequent lymph node sample and repeating step (b) when the level of the one or more lymph node sufficiency biomarkers in a previous sample, optionally an immediately preceding sample, indicates insufficiency.

5. The method of claim 4, wherein the lymph node sample and the subsequent samples are taken from a same lymph node.

6. The method of any of the preceding claims, wherein the one or more lymph node sufficiency biomarkers is or comprises CXCL13.

7. The method of any of the preceding claims, wherein the one or more lymph node sufficiency biomarkers is or comprises CCL21.

8. The method of any of the preceding claims, wherein the one or more lymph node sufficiency biomarkers further comprises SIGLEC1.

9. The method of any of the preceding claims, wherein the one or more lymph node sufficiency biomarkers further comprises UBD.

10. The method of any one of claims 1-7, wherein the one or more lymph node sufficiency biomarkers is or comprises CXCL13 and CCL21.

11. The method of any one of claims 1-7, wherein the one or more lymph node sufficiency biomarkers is or comprises CXCL13, CCL21 and SIGLEC1.

12. The method of any one of claims 1-7, wherein the one or more lymph node sufficiency biomarkers is or comprises CXCL13, CCL21 and UBD.

13. The method of any one of claims 1-7, wherein the one or more lymph node sufficiency biomarkers is or comprises CXCL13, CCL21, SIGLEC1 and UBD.

14. The method of any of the preceding claims, wherein the sample is a biopsy sample.

15. The method of claim 14, wherein the biopsy sample is a needle aspirate sample.

16. The method of claim 15, wherein the needle aspirate sample is an endobronchial ultrasound-guided transbronchial needle aspiration (EBUS-TBNA) sample.

17. The method of any one of claims 1-16, wherein for a sample determined to be sufficient, the method further comprises subjecting the sample to an assay, optionally next generation sequencing, ROSE and/or a pathological assay and/or assessment.

18. The method of any of the preceding claims, further comprising performing an assay or method to detect metastasis.

19. The method of claim 18, wherein the assay for detecting metastasis comprises measuring a level of one or more lymph node metastasis biomarkers selected from the group consisting of EpCAM, KRT19, SFN, and/or KRT7 in the sample; wherein the level of the one or more lymph node metastasis biomarkers is indicative of metastasis.

20. The method of claim 19, wherein the group of lymph node metastasis biomarkers for metastasis further comprises INSM1 and KRT5.

21. The method of claim 19 or 20, wherein the one or more lymph node metastasis biomarkers are at least 2 lymph node biomarkers for metastasis.

22. The method of any one of claims 19-21, wherein the one or more lymph node metastasis biomarkers are at least 3 lymph node biomarkers for metastasis.

23. The method of any one of claims 19-22, wherein the one or more lymph node metastasis biomarkers are at least 4 lymph node biomarkers for metastasis.

24. The method of any one of claims 19-23, wherein the one or more lymph node metastasis biomarkers are at least 5 lymph node biomarkers for metastasis.

25. The method of any one of claims 19-24, wherein the one or more lymph node metastasis biomarkers are the 6 lymph node biomarkers for metastasis.

26. The method of any one of claims 19-25, wherein the one or more lymph node metastasis biomarkers is or comprises EpCAM.

27. The method of any one of claims 19-26, wherein the one or more lymph node metastasis biomarkers is or comprises KRT19.

28. The method of any one of claims 19-27, wherein the one or more lymph node metastasis biomarkers is or comprises SFN.

29. The method of any one of claims 19-28, wherein the one or more lymph node metastasis biomarkers is or comprises KRT7.

30. The method of any one of claims 19-29, wherein the one or more lymph node metastasis biomarkers is or comprises INSM1.

31. The method of any one of claims 19-30, wherein the one or more lymph node metastasis biomarkers is or comprises KRT5.

32. The method of any one of claims 19-31, further comprising comparing the level of the one or more metastasis lymph node biomarkers to a predetermined cut-off value or set of predetermined cut-off values.

33. The method of claim 32, wherein the predetermined cut-off value or set of predetermined cut-off values is determined from a plurality of non-metastatic lymph node samples.

34. The method of any one of claims 1 to 17, wherein the method is followed by the method of any one of claims 18-33, when the sample is determined to be sufficient.

35. The method of any one of claims 1-33, wherein determining sample sufficiency and detecting metastasis are performed concurrently.

36. The method of any one of claims 1-35, wherein the sample is from a subject suspected of having lung cancer.

37. The method of any one of claims 1-35, wherein the sample is from a subject diagnosed as having lung cancer.

38. The method of claim 36 or 37, wherein the lung cancer is adenocarcinoma, non-small cell lung cancer, small cell lung cancer or squamous cell lung cancer.

39. The method of any one of claims 1-38, wherein the sample is a protein fraction.

40. The method of claim 39, wherein the level of the one or more lymph node biomarkers is measured using in an affinity assay using a binding agent.

41. A method of detecting metastasis, the method comprising: providing a lymph node sample obtained from a subject, measuring a level of one or more lymph node metastasis biomarkers selected from the group consisting of EpCAM, KRT19, SFN, and KRT7 in the sample; wherein the level of the one or more lymph node metastasis biomarkers is indicative of metastasis.

42. The method of claim 41, wherein the group of lymph node metastasis biomarkers further comprises INSM1 and KRT5.

43. The method of claim 41 or 42, wherein the one or more lymph node metastasis biomarkers are at least 2 lymph node biomarkers for metastasis.

44. The method of any one of claims 41-43, wherein the one or more lymph node metastasis biomarkers are at least 3 lymph node biomarkers for metastasis.

45. The method of any one of claims 41-44, wherein the one or more lymph node metastasis biomarkers are at least 4 lymph node biomarkers for metastasis.

46. The method of any one of claims 41-45, wherein the one or more lymph node metastasis biomarkers are at least 5 lymph node biomarkers for metastasis.

47. The method of any one of claims 41-46, wherein the one or more lymph node metastasis biomarkers are the 6 lymph node biomarkers for metastasis.

48. The method of any one of claims 41-47, wherein the one or more lymph node metastasis biomarkers is or comprises EpCAM.

49. The method of any one of claims 41-48, wherein the one or more lymph node metastasis biomarkers is or comprises KRT19.

50. The method of any one of claims 41-49, wherein the one or more lymph node metastasis biomarkers is or comprises SFN.

51. The method of any one of claims 41-50, wherein the one or more lymph node metastasis biomarkers is or comprises KRT7.

52. The method of any one of claims 41-51, wherein the one or more lymph node metastasis biomarkers is or comprises INSM1.

53. The method of any one of claims 41-52, wherein the one or more lymph node metastasis biomarkers is or comprises KRT5.

54. The method of any one of claims 41-53, further comprising comparing the level of the one or more lymph node metastasis biomarkers to a predetermined cut-off value or set of predetermined cut-off values.

55. The method of claim 54, wherein the predetermined cut-off value or set of predetermined cut-off values is determined from a plurality of non-metastatic lymph node samples.

56. The method of any one of claims 41-55, wherein the sample is a biopsy sample.

57. The method of claim 56, wherein the biopsy sample is a needle aspirate sample.

58. The method of claim 57, wherein the needle aspirate sample is an endobronchial ultrasound-guided transbronchial needle aspiration (EBUS-TBNA) sample.

59. The method of any one of claims 41-58, wherein the sample is from a subject suspected of having lung cancer.

60. The method of any one of claims 41-58, wherein the sample is from a subject diagnosed as having lung cancer.

61. The method of claim 59 or 60, wherein the lung cancer is adenocarcinoma, non-small cell lung cancer, small cell lung cancer or squamous cell lung cancer.

62. The method of any one of claims 41-61, wherein the sample is a protein fraction.

63. The method of claim 62, wherein the level of the one or more lymph node biomarkers is measured using in an affinity assay using a binding agent.

64. The method of any one of claims 41-61, wherein the sample is a nucleic acid sample.

65. The method of claim 64, wherein the level of the one or more lymph node biomarkers is measured using a hybridization assay using a nucleic acid or peptide nucleic acid probe.

66. A device, optionally a point-of-care (POC) device, comprising:
a solid support, comprising a binding agent for CXC13 and/or a binding agent for CCL21, optionally further comprises a binding agent for SIGLEC1 and/or a binding agent for UBD; optionally wherein the device is for determining sufficiency in a lymph node sample.

67. The device of claim 66, wherein the solid support further comprises a binding agent for each of at least 2, at least 3, at least 4, at least 5 of EpCAM, KRT19, SFN, KRT7, INSM1 and KRT5; optionally wherein the device is for detecting metastasis in a lymph node sample.

68. The device of claim 66 or 67, wherein the device comprises an immunological assay.

69. The device of claim 68, wherein the immunological assay is an enzyme-linked immunosorbent assay (ELISA) or a lateral flow assay (LFA).

70. The device of claim 69, wherein the immunological assay is a lateral flow assay (LFA).

71. The device of any one of claims 66-70, wherein the binding agents are fluorescently labelled and/or antibodies or monobodies.

72. The device of any one of claims 66-71, wherein the device is for use in the method of any one of claims 1-38.

73. A device, optionally a point-of-care (POC) device, comprising:
a solid support, comprising a binding agent for each of at least 2, at least 3, at least 4 or at least 5 of EpCAM, KRT19, SFN, KRT7, INSM1 and/or KRT5, optionally for detecting metastasis in a lymph node sample.

74. The device of claim 73, wherein the device comprises a PCR-based assay.

75. The device of claim 73, wherein the device comprises an immunological assay.

76. The device of claim 75, wherein the immunological assay is an enzyme-linked immunosorbent assay (ELISA) or lateral flow assay (LFA).

77. The device of claim 76, wherein the immunological assay is a lateral flow assay (LFA).

78. The device of any one of claims 73-76, wherein the binding agents are fluorescently labelled.

79. The device of claim 73, wherein the device comprises an electrochemical assay.

80. The device of any one of claims 73-79, wherein the device is for use in the method of any one of claims 41-61.

81. The method of any one of claims 1-13, wherein the sample is a needle rinse sample.
